(19) United States
(12) Patent Application Publication
(10) Pub. No.: US 2017/0210840 A1
(43) Pub. Date: Jul. 27, 2017

(54) THERMOGELLING GRAFT COPOLYMER AND METHOD OF PREPARATION THEREOF

(71) Applicant: National University of Singapore, Singapore (SG)

(72) Inventors: Jun LI, Singapore (SG); Bishuo DIAO, Singapore (SG)

(21) Appl. No.: 15/500,387

(22) PCT Filed: Aug. 19, 2015
(86) PCT No.: PCT/SG2015/050267

§ 371 Date: Jan. 30, 2017

(30) Foreign Application Priority Data
Aug. 19, 2014 (SG) 10201405015U

(51) Int. Cl. C08F 293/00; A61K 9/00; C08F 289/00; A61K 47/32; C08H 7/00; C08F 285/00

(52) U.S. Cl. CPC C08F 293/005; C08H 6/00; C08F 285/00; C08F 289/00; A61K 47/32; A61K 9/0024; C08F 2438/01

(57) ABSTRACT

A thermogelling graft copolymer is provided. The copolymer is obtainable by (a) polymerizing, in a first step, an activated lignin-based polymer with N-isopropylacrylamide (NIPAAm) to form a lignin/poly(N-isopropylacrylamide) (PNIPAAm) graft copolymer, and (b) polymerizing, in a second step, the lignin/poly(N-isopropylacrylamide) (PNIPAAm) graft copolymer with a monomer mixture comprising (i) poly(ethylene glycol) (meth)acrylate and/or a C₁₋₁₀ alkyl ether thereof, and (ii) poly(propylene glycol) (meth)acrylate and/or a C₁₋₁₀ alkyl ether thereof, to obtain a lignin-poly(N-isopropylacrylamide)-block-poly(ethylene glycol)/poly(propylene glycol) graft copolymer. Method of preparing the thermogelling graft copolymer is also provided.

### CROSS-REFERENCE TO RELATED APPLICATION

This application claims the benefit of priority of Singapore patent application No. 10201405015U filed on 19 Aug. 2014, the content of which is incorporated herein by reference in its entirety for all purposes.

### TECHNICAL FIELD

Various embodiments relate to a thermogelling graft copolymer and method of preparing the thermogelling graft copolymer.

### BACKGROUND

Lignin is an organic substance binding cells, fibres and vessels which constitute wood and the lignified elements of plants, as in straw. It is present in all vascular plants and constitutes from about a quarter to a third of the dry mass of wood. It belongs to the class of natural renewable polymer, and is the second most abundant biomass next to cellulose. Together with hemicelluloses and cellulose, they constitute the principal components of wood and annual plants.

Lignin comprises a dendritic network polymer of phenyl propene basic units. It is made of three basic monomer types, namely, paracoumaryl alcohol, coniferyl alcohol, and sinapyl alcohol. These monomers are incorporated in lignin polymers in the form of phenylpropanoids p-hydroxyphenyl (H), guaiacyl (G), and syringyl (S). The monomer alcohols are able to link one another via oxidative coupling to generate complex, amorphous three-dimensional polymer networks. As all three types of lignin monomers possess a hydroxyl group in the aliphatic side chain and a phenol group in the aromatic ring, this allows modification of lignin via the hydroxyl and phenol groups.

Current utilization of lignin is largely constrained apart from its traditional use as an energy source and for leather tanning purposes, as lignin is a highly difficult and challenging material to work with. With increasing emphasis being placed upon safeguarding the environment, greater attention has been directed towards utilizing such abundant biomass.

Although functional plastic materials have been produced from lignin, these materials are generally thermosets containing permanent chemically cross-linked networks, which are not melt-processable, and hence not recyclable. Furthermore, due to their rigid and brittle character, the lignin-based plastics generally lack the rubber elasticity, strength, and toughness required for use in many industrial and commercial applications.

Of particular importance are gels made from lignin. Early attempts to synthesize lignin-based gels include soft gels derived from (a) technical lignin for separation of alcohol molecules from fermentation broths, and (b) thermoplastic that is made of a blend of lignin with other hydrophilic polymers like poly(ethylene oxide). As in the case for lignin-based thermosets which possess permanent, highly-cross linked network structures, physical cross-linked hydrogels that undergo reversible phase transition in response to external stimuli such as changes in pH and temperature show much promise for biomedical applications. Advantageously, the fact that in situ-forming hydrogels are injectable fluids prior to administration into a human or animal body, but take shape immediately within the tissue or body cavity, eliminates the need for surgical placement of the hydrogels.

Prior attempts to synthesize lignin-based copolymers usually employ free radical polymerization. Some lignin-based copolymers were derived using condensation polymerization. There were also some reports on utilization of chemo-enzymatic approach.

Notwithstanding the above, there remains a need for improved materials formed from lignin and methods to synthesize such lignin-based materials in a more efficient manner.

### SUMMARY

In a first aspect, a thermogelling graft copolymer is provided. The copolymer is obtainable by
(a) polymerizing, in a first step, an activated lignin-based polymer with N-isopropylacrylamide (NIPAAm) to form a lignin/poly(N-isopropylacrylamide) (PNIPAAm) graft copolymer, and
(b) polymerizing, in a second step, the lignin/poly(N-isopropylacrylamide) (PNIPAAm) graft copolymer with a monomer mixture comprising (i) poly(ethylene glycol) (meth)acrylate and/or a C₁₋₁₀ alkyl ether thereof, and (ii) poly(propylene glycol) (meth)acrylate and/or a C₁₋₁₀ alkyl ether thereof, to obtain a lignin-poly(N-isopropylacrylamide)-block-poly(ethylene glycol)/poly(propylene glycol) graft copolymer.

In a second aspect, a method of preparing a thermogelling graft copolymer according to the first aspect is provided. The method comprises
a) reacting a lignin-based polymer with an atom transfer radical polymerization (ATRP) initiator to form an activated lignin-based polymer;
b) polymerizing N-isopropylacrylamide (NIPAAm) with the activated lignin-based polymer by ATRP to form a poly(N-isopropylacrylamide)-grafted lignin-based polymer, and
c) polymerizing the poly(N-isopropylacrylamide)-grafted lignin-based polymer with a monomer mixture comprising (i) poly(ethylene glycol) (meth)acrylate and/or a C₁₋₁₀ alkyl ether thereof, and (ii) poly(propylene glycol) (meth)acrylate and/or a C₁₋₁₀ alkyl ether thereof, to obtain the lignin-poly(N-isopropylacrylamide)-block-poly(ethylene glycol)/poly(propylene glycol) graft copolymer.

In a third aspect, a composition comprising a thermogelling graft copolymer according to the first aspect and a therapeutic agent is provided.

### BRIEF DESCRIPTION OF THE DRAWINGS

The invention will be better understood with reference to the detailed description when considered in conjunction with the non-limiting examples and the accompanying drawings, in which:

FIG. 1 is a schematic diagram showing synthesis route of lignin-based copolymers (LnNEPs) according to embodiments disclosed herein.

FIG. 2 shows (A) UV-vis spectra for lignin of different concentrations, and (B) calibration curve for lignin content computation.

FIG. 3 shows (A) UV-vis spectra for lignin macro-initiators (MIs) at 0.025 mg/mL, and (B) UV-vis spectra for lignin-based copolymers (LnNEPs) at 0.125 mg/mL.

FIG. 4 shows ¹H NMR spectrum of LnNEP-5% in CDCl₃.

FIG. 5 shows plots of average diameters of the lignin-based copolymers against different temperatures for (A) LnNEP copolymers, and (B) LnNEP-5% and control copolymers.

FIG. 6 shows photographs depicting LnNEP-5% (7.0 wt % in H₂O) at different temperatures of (a) room temperature (25° C.); (b) 33° C.; (c) 35° C.; and (d) 50° C.

FIG. 7 shows sol-gel phase diagrams of LnNEP copolymers in comparison with control copolymers.

FIG. 8 shows dynamic rheological analysis of (A) LnNEP copolymers (at 7.0 wt %), and (B) LnNEP-5%, LnNE-5%, and LnEP-5% copolymers (at 2.5 wt %) in aqueous medium at different temperatures.

FIG. 9 is a graph showing Doxorubicin release profiles of LnNEP and LnNE copolymer hydrogels (7 wt %) in PBS.

### DETAILED DESCRIPTION

In a first aspect, a thermogelling graft copolymer is provided. The term “thermogelling” as used herein refers to the property or characteristic of a liquid or solution to turn into a non-liquid such as a gel in response to temperature changes. The lowest temperature at which some gelation of a material is observed is termed as the “critical gelation temperature”. Accordingly, gelation may be effected at the critical gelation temperature or at temperatures above the critical gelation temperature.

The term “graft copolymer” as used herein refers to a copolymer having a backbone or main chain to which side chains of a different chemical composition are attached at various positions along the backbone. For example, the backbone may be formed of a first polymer and the side chains of a second polymer, wherein the first polymer and the second polymer have different chemical compositions. The side chains may be grafted or attached at various positions along the backbone by covalent bonding to form the graft copolymer.

The thermogelling graft copolymer is obtainable or may be obtained by polymerizing, in a first step, an activated lignin-based polymer with N-isopropylacrylamide (NIPAAm) to form a lignin/poly(N-isopropylacrylamide) (PNIPAAm) graft copolymer, and by polymerizing, in a second step, the lignin/poly(N-isopropylacrylamide) (PNIPAAm) graft copolymer obtained in the first step with a monomer mixture comprising (i) poly(ethylene glycol) (meth)acrylate and/or a C₁₋₁₀ alkyl ether thereof, and (ii) poly(propylene glycol) (meth)acrylate and/or a C₁₋₁₀ alkyl ether thereof. In so doing, a lignin-poly(N-isopropylacrylamide)-block-poly(ethylene glycol)/poly(propylene glycol) graft copolymer is obtained.

In the context of the present application, the lignin-based polymer forms the core or backbone of the thermogelling graft copolymer, with multiple arms of polymer chains as side chains grafted thereon. In various embodiments, each side chain may include a block of PNIPAAm and a block of brush-like random copolymer of poly(ethylene glycol) (PEG) and poly(propylene glycol) (PPG), such as that shown in formula (I).

As mentioned above, the thermogelling graft copolymer may be obtained by polymerizing, in a first step, an activated lignin-based polymer with N-isopropylacrylamide (NIPAAm) to form a lignin/poly(N-isopropylacrylamide) (PNIPAAm) graft copolymer.

The activated lignin-based polymer is obtainable or may be obtained by reacting a lignin-based polymer with an atom transfer radical polymerization (ATRP) initiator.

The lignin-based polymer may be a polymer blend containing lignin, and/or a mixture containing lignin as one of the components.

The lignin may be a sulfur-bearing lignin, such as lignosulphonates, kraft lignins, or combinations thereof. As used herein, the term “kraft lignin” refers to lignin material which is typically recovered from a kraft process. The kraft process may otherwise be known as kraft pulping or sulfate process, refers to a process for conversion of wood into wood pulp containing almost pure cellulose fibers. Generally, it involves treatment of wood chips with a mixture of sodium hydroxide and sodium sulfide which breaks the bonds that link lignin to the cellulose.

Advantageously, the polyphenolic nature of lignin and its low toxicity, along with its desirable properties such as its dispersing, binding, complexing and emulsifying, thermal stability, specific UV-absorbing, water repelling, and conductivity characteristics, render its attractiveness as a renewable replacement for toxic and expensive fossil fuel-derived polymer feedstocks. Furthermore, lignin is biodegradable and is one of the most durable biopolymers available.

Lignin may be present in the thermogelling graft copolymer in an amount in the range of about 5 wt % to about 15 wt %, such as about 5 wt % to about 12 wt %, about 5 wt % to about 10 wt %, about 5 wt % to about 8 wt %, about 8 wt % to about 15 wt %, about 10 wt % to about 15 wt %, about 12 wt % to about 15 wt %, about 8 wt % to about 12 wt %, about 7 wt % to about 13 wt %, or about 9 wt % to about 14 wt %.

In various embodiments, the lignin-based polymer has a number average molecular weight (Mₑ) in the range of about 3000 g/mol to about 7000 g/mol. The number average molecular weight may be obtained by dividing the weight of a sample by the number of molecules of which it is composed. For example, the lignin-based polymer may have a number average molecular weight in the range of about 3000 g/mol to about 6000 g/mol, such as about 3000 g/mol to about 5000 g/mol, about 3000 g/mol to about 4000 g/mol, about 4000 g/mol to about 7000 g/mol, about 5000 g/mol to about 7000 g/mol, about 4000 g/mol to about 6000 g/mol, or about 4500 g/mol to about 5500 g/mol. In specific embodiments, the lignin-based polymer has a number average molecular weight of about 5000 g/mol.

As mentioned herein, the lignin-based polymer is reacted with an atom transfer radical polymerization (ATRP) initiator to form an activated lignin-based polymer. The activated lignin-based polymer may be a polymer conjugate containing lignin and an ATRP initiator. In such a configuration, the lignin may form a backbone to which an ATRP initiator may be attached.

In various embodiments, the atom transfer radical polymerization (ATRP) initiator comprises or consists of 2-bromoisobutyryl bromide.

Between about 5% to about 40% of hydroxyl groups in the lignin-based polymer may be modified into macro-initiation sites in the activated lignin-based polymer. For example, proportion of hydroxyl groups in the lignin-based polymer that are modified into macro-initiation sites in the activated lignin-based polymer may be in the range of about 5% to about 35%, such as about 5% to about 30%, about 5% to about 25%, about 5% to about 20%, about 5% to about 15%, about 5% to about 10%, about 10% to about 40%, about 15% to about 40%, about 20% to about 40%, about 25% to about 40%, about 30% to about 40%, about 15% to about 25%, or about 10% to about 30%.

Lignin content in the activated lignin-based polymer may be in the range of about 65 wt % to about 95 wt %, such as about 65 wt % to about 90 wt %, about 65 wt % to about 85 wt %, about 65 wt % to about 80 wt %, about 65 wt % to about 75 wt %, about 70 wt % to about 95 wt %, about 75 wt % to about 95 wt %, about 80 wt % to about 95 wt %, about 75 wt % to about 85 wt %, or about 70 wt % to about 90 wt %.

The activated lignin-based polymer may be dissolved in a suitable solvent, such as N,N-dimethylacetamide, dimethylformamide, dimethyl sulfoxide, alcohols, acetone, acetonitrile, dioxane, tetrahydrofuran, and/or water.

The activated lignin-based polymer is polymerized with N-isopropylacrylamide (NIPAAm) by atom transfer radical polymerization (ATRP) to form a lignin/poly(N-isopropylacrylamide) (PNIPAAm) graft copolymer.

Atom transfer radical polymerization (ATRP) is a recently developed ‘living’ radical polymerization method, involving a copper halide/nitrogen-based ligand catalyst. Advantageously, the method does not require stringent experimental conditions, as in the cases of cationic and anionic polymerization. This controlled radical polymerization technique allows for the polymerization and block-copolymerization of a wide range of functional monomers in a controlled fashion, yielding polymers with narrowly dispersed molecular weights, predetermined by the concentration ratio of the consumed monomer to initiator.

In various embodiments, the ATRP reaction is carried out in an inert environment, such as in a nitrogen atmosphere, and may take place for a time period in the range of hours, such as about 24 hours to about 60 hours, or about 36 hours to about 48 hours. Advantageously, the reaction may be carried out at room temperature. In various embodiments, the polymerization is carried out in the presence of a copper halide/nitrogen-based ligand catalyst. For example, the catalyst may comprising a complex formed from a copper halide, such as Cu(I)Br, and 1,1,4,7,10,10-hexamethyltriethylenetetramine.

As mentioned above, the lignin-based polymer forms the backbone of the graft copolymer. By carrying out ATRP, poly(N-isopropylacrylamide) is grafted as a side chain on the lignin-based polymer backbone.

The lignin/poly(N-isopropylacrylamide) (PNIPAAm) graft copolymer obtained is reacted with a monomer mixture comprising (i) poly(ethylene glycol) (meth)acrylate and/or a C₁₋₁₀ alkyl ether thereof, and (ii) poly(propylene glycol) (meth)acrylate and/or a C₁₋₁₀ alkyl ether thereof. The lignin/poly(N-isopropylacrylamide) (PNIPAAm) graft copolymer is able to function as an ATRP initiator during polymerization. In so doing, a lignin-poly(N-isopropylacrylamide)-block-poly(ethylene glycol)/poly(propylene glycol) graft copolymer is obtained.

As in the case for forming poly(N-isopropylacrylamide)-grafted lignin-based polymer, the ATRP reaction to form the lignin-poly(N-isopropylacrylamide)-block-poly(ethylene glycol)/poly(propylene glycol) graft copolymer may be carried out in an inert environment, such as in a nitrogen atmosphere, using a similar catalyst such as a complex formed from Cu(I)Br and 1,1,4,7,10,10-hexamethyltriethylenetetramine.

The ATRP reaction to form the lignin-poly(N-isopropylacrylamide)-block-poly(ethylene glycol)/poly(propylene glycol) graft copolymer may take place for a time period in the range of hours, such as about 12 hours to about 48 hours, about 12 hours to about 36 hours, or about 12 hours to about 24 hours. The reaction may be carried out at a temperature in the range of about 30° C. to about 80° C., such as about 50° C. to about 80° C., about 60° C. to about 80° C., about 70° C. to about 80° C., about 50° C. to about 70° C., or about 40° C. to about 70° C.

The thermogelling graft copolymer comprises or consists of a lignin-poly(N-isopropylacrylamide)-block-poly(ethylene glycol)/poly(propylene glycol) graft copolymer.

As used herein, the term “block copolymer” refers to a copolymer containing a linear arrangement of blocks, wherein “block” may be defined as a portion of a polymer molecule in which the monomeric units have at least one constitutional or configurational feature absent from the adjacent portions. For example, the block copolymer may contain two or more types of blocks, such as di-block for a block copolymer containing two types of blocks; tri-blocks for a block copolymer containing three types of blocks, tetra-blocks for a block copolymer containing four types of blocks, and so on. By way of illustration only, a block copolymer of moiety P and moiety Q may be denoted -P-P-Q-Q-Q-, and may accordingly be termed an “PQ block copolymer” or a “di-block copolymer”. Other examples include, but are not limited to, PQP block copolymers (-P-P-Q-Q-P-P-) and PQR block copolymers (-P-P-P-Q-Q-Q-R-R-R-).

In various embodiments, the at least one block copolymer comprises blocks of (i) at least one poly(N-isopropylacrylamide) block, and (ii) at least one poly(ethylene glycol)/poly(propylene glycol) copolymer block, wherein the at least one poly(ethylene glycol)/poly(propylene glycol) copolymer block is grafted to the lignin-based polymer via the at least one poly(N-isopropylacrylamide) block.

It is specified herein that lignin-poly(N-isopropylacrylamide)-random-poly(ethylene glycol)/poly(propylene glycol) graft copolymers do not form part of the invention, and it was surprisingly found by the inventors that lignin-poly(N-isopropylacrylamide)-random-poly(ethylene glycol)/poly(propylene glycol) graft copolymers do not form hydrogels at any temperature, and solutions containing the graft copolymer only turned from transparent to turbid when temperature was increased.

In specific embodiments, the thermogelling graft copolymer is represented by formula (I)

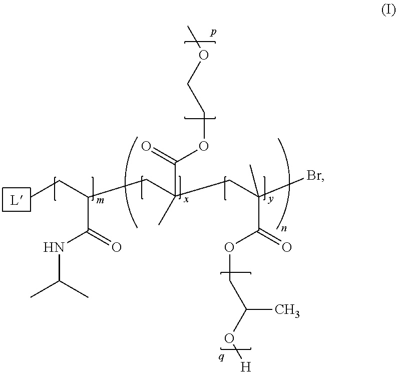

wherein L′ is the lignin-based polymer, and m, n, x, y, p and q are independently integers in the range of about 0 to 10000.

In various embodiments, m, n, x, y, p and q are independently integers in the range of about 1 to about 10000, such as about 10 to about 10000, about 100 to about 10000, about 1000 to about 10000, about 5000 to about 10000, about 0 to about 100, about 0 to about 1000, about 0 to about 5000, about 5 to about 8000, about 5 to about 5000, about 5 to about 2000, or about 10 to about 1000. In preferred embodiments, m, n, x, y, p and q are independently integers in the range of about 5 to about 500.

Poly(ethylene glycol) (PEG) and polypropylene glycol) (PPG) are biocompatible and clearance from the body is possible for blocks of lower molecular weight, for example about 10,000 g/mol. Advantageously, PEG provides hydrophilic blocks that may absorb and retain large quantities of water, while PPG is highly thermosensitive and provides a balanced hydrophobicity and hydrophilicity at different temperatures, facilitating formation of a thermosensitive hydrogel.

Weight ratio of N-isopropylacrylamide monomer to the monomer mixture comprising (i) poly(ethylene glycol) (meth)acrylate and/or a C₁₋₁₀ alkyl ether thereof, and (ii) poly(propylene glycol) (meth)acrylate and/or a C₁₋₁₀ alkyl ether thereof, may be in the range of about 1:1 to about 2:1, such as about 5:4 to about 2:1, about 6:5 to about 3:2, about 3:2 to about 2:1, or about 1:1 to about 3:2.

As mentioned above, “thermogelling” refers to the property or characteristic of a liquid to turn into a non-liquid such as a gel in response to temperature changes. The thermogelling graft copolymer disclosed herein is able to solidify or convert from a liquid to a gel in response to an increase in temperature at the critical gelation temperature or at temperatures above the critical gelation temperature.

In various embodiments, the lignin-poly(N-isopropylacrylamide)-block-poly(ethylene glycol)/poly(propylene glycol) graft copolymer forms a hydrogel at a temperature in the range of about 32° C. to about 50° C. For example, the thermogelling graft copolymer may form a hydrogel at a temperature in the range of about 32° C. to about 45° C., about 32° C. to about 40° C., about 32° C. to about 38° C., about 32° C. to about 35° C., about 35° C. to about 50° C., about 38° C. to about 50° C., about 40° C. to about 50° C., about 42° C. to about 50° C., about 45° C. to about 50° C., about 38° C. to about 45° C., or about 33° C. to about 38° C.

In some embodiments, the thermogelling of the thermogelling graft copolymer is induced by physiological temperature, such as around that of the human body (37° C.).

The thermogelling graft copolymer may have a critical gelation concentration in the range of about 1 wt % to about 5 wt % in solution. As used herein, the term “critical gelation concentration” refers to the minimum copolymer concentration required in aqueous solution before gelation behavior may be observed. A lower critical gelation concentration is preferred, as lower concentrations of polymer may be used to create a gel. This is advantageous, for example, in applications which involves implantation of the thermogelling graft copolymer in a subject, since smaller amounts of polymer are being used. In various embodiments, the thermogelling graft copolymer has a critical gelation concentration in the range of about 1 wt % to about 4 wt %, such as about 1 wt % to about 3 wt %, about 2 wt % to about 4 wt %, or about 1 wt % to about 2.5 wt % in solution. In specific embodiments, the thermogelling graft copolymer has a critical gelation concentration in the range of about 1 wt % to about 3 wt % in solution.

Various embodiments refer in a second aspect to a method of preparing a thermogelling graft copolymer according to the first aspect. The method comprises (a) reacting a lignin-based polymer with an atom transfer radical polymerization (ATRP) initiator to form an activated lignin-based polymer; (b) polymerizing N-isopropylacrylamide (NIPAAm) with the activated lignin-based polymer by ATRP to form a poly(N-isopropylacrylamide)-grafted lignin-based polymer, and (c) polymerizing the poly(N-isopropylacrylamide)-grafted lignin-based polymer with a monomer mixture comprising (i) poly(ethylene glycol) (meth)acrylate and/or a C₁₋₁₀ alkyl ether thereof, and (ii) poly(propylene glycol) (meth)acrylate and/or a C₁₋₁₀ alkyl ether thereof, to obtain the lignin-poly(N-isopropylacrylamide)-block-poly(ethylene glycol)/poly(propylene glycol) graft copolymer.

Details of the method of preparing the thermogelling graft copolymer, such as reaction conditions, and ratio of reactants, for example, have already been discussed above.

Various embodiments refer in a further aspect to a composition comprising a thermogelling graft copolymer according to the first aspect and a therapeutic agent.

The therapeutic agent may be any compound that is to be delivered to cells in culture or is to be delivered within the body of a subject. Advantageously, the therapeutic agents may be incorporated in the solution state at low temperatures, which may then be injected in vivo, where the higher body temperature induces formation of a gel. The injected gel may be used for the controlled release of the therapeutic or bioactive agents. Biodegradability of the polymers is advantageous, since degradation of the polymer into smaller fragments allows for subsequent removal of the polymer from the body.

Examples of therapeutic agent include a nucleic acid, such as DNA, a peptide, a protein, a small molecule, a cell, an antibody, an antigen, a ligand, a hormone, a growth factor, a cell signalling molecule, a cytokine, an enzyme inhibitor, an antibiotic, a chemotherapeutic agent, an anti-inflammatory agent, and/or an analgesic. In specific embodiments, the therapeutic agent comprises or consists of an anti-cancer drug doxorubicin.

In various embodiments, the composition has a critical gelation temperature below 37° C. Compositions which change state at about this temperature are useful, because they will remain in a body cavity, for example, after they have been delivered, as opposed to that of a liquid, which would have difficulty in remaining in place in the body due to movement of the subject.

The composition may further comprise a pharmaceutically acceptable diluent or carrier. In general, a diluent or carrier is selected on the basis of the mode and route of administration, and standard pharmaceutical practice.

In further embodiments, the composition may comprise a solvent, such as an aqueous solvent.

A method of delivering a therapeutic agent to a subject is also disclosed herein. The method comprises delivering a composition of the third aspect to the body of a subject.

The composition may be a liquid prior to delivering to the body and delivering may comprise injection. After injection, the thermogelling composition along with the active ingredients may become a gel. As mentioned above, this gel may come into contact with body fluid to allow sustained release of the active ingredients. In so doing, the injection frequency may be lowered, thereby improving patient compliance, which is especially important for drugs currently only available through injection, such as protein/peptide (e.g. insulin and growth hormones) drugs.

Concentration of therapeutic agent to be included in the composition may vary depending on the particular therapeutic agent, the site of delivery, the age, weight and sex of the subject and the particular reason for delivery of the therapeutic agent and may be routinely determined by one skilled in the art. The subject may be any subject that is in need of receiving the therapeutic agent, including a mammal, including a human subject. Compositions may be prepared to contain an effective amount or an amount that is sufficient to achieve the desired effect.

The thermogelling graft copolymer disclosed herein may also be used for the manufacture of a medicament for delivering a therapeutic agent to the body of a subject. Therapeutic compositions containing the thermogelling copolymer disclosed herein may be used on the body, for example, mucosal surface location of the body such as in rectal, vaginal, oral cavity, ophthalmic and nasal locations. It may also be used a topical and transdermal composition and as an injectable, in situ gelling composition for subcutaneous or intramuscular applications to deliver specific medicaments where needed.

Hereinafter, the present invention will be described more fully with reference to the accompanying drawings, in which exemplary embodiments of the invention are shown. This invention may, however, be embodied in many different forms and should not be construed as limited to the exemplary embodiments set forth herein. Rather, these embodiments are provided so that this disclosure will be thorough and complete, and will fully convey the scope of the invention to those skilled in the art. In the drawings, lengths and sizes of layers and regions may be exaggerated for clarity.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### EXPERIMENTAL SECTION

In various embodiments, a series of lignin-based thermogelling graft copolymers was synthesized using ATRP technique. The lignin-based thermogelling graft copolymers are composed of a lignin core and multiple arms of graft polymer chains, where each graft consists of a block of PNIPAAm and a block of brush-like random copolymer of poly(ethylene glycol) (PEG) and poly(propylene glycol) (PPG). A few control copolymers were also synthesized for comparison purpose. The thermo-responsiveness of the lignin-based copolymers was investigated using dynamic light scattering, and the thermogelling properties of the copolymers were demonstrated by sol-gel transition and rheological studies. Finally, the hydrogels of the copolymers were tested for sustained release of anti-cancer drug doxorubicin.

It was found that the lignin-based PNIPAAm-block-PEG/PPG copolymers were thermo-responsive in aqueous medium. The aqueous solutions of the copolymers displayed thermogelling behaviors, turning from sol at low temperatures to hydrogel at temperatures around 32-34° C., and further to dehydrated gel at higher temperatures. The thermogelling copolymers had very low critical gelation concentrations ranging from 1.3 to 2.5 wt %. The elastic modulus G′ and viscous modulus G″ of the copolymer solutions were very low at low temperatures, and increased at higher temperatures, and then crossed over at their gelation temperatures, with G′ dominating G″ after the gel formation. The block architecture of the lignin-based PNIPAAm-block-PEG/PPG copolymers is a key for the hydrogel formation, because the lignin-based PNIPAAm-random-PEG/PPG copolymer with the same monomer ratio, as a control polymer, could not form hydrogels at any temperature, whose solution only turned from transparent to turbid when the temperature was increased.

The lignin-based copolymers showed thermogelling transition at a temperature above room temperature but below human body temperature, and could potentially be useful in biomedical applications such as for injectable controlled drug release.

### Example 1: Materials

Kraft lignin (alkali, Mₙ=5,000, M_{w}=28,000) was purchased from Sigma-Aldrich. It was vacuum dried at 90° C. overnight before use. Poly(ethylene glycol) methyl ether methacrylate (PEGMEMA, Mₙ=475), poly(propylene glycol) methacrylate (PPGMA, Mₙ=375), and N-isopropylacrylamide (NIPAAm, M_{w}=113.16) were purchased from Sigma-Aldrich, and used as received. N,N-Dimethylformamide (DMF, anhydrous, 99.8%), N,N-dimethylacetamide (DMA, anhydrous, 99.8%), 1,4-dioxane (anhydrous, 99.8%), 2-bromoisobutyryl bromide (BiBB), 1,1,4,7,10,10-hexamethyl-triethylenetetramine (HMTETA) (97.0%), diethyl ether, and copper(I) bromide (CuBr, >98.0%) were purchased from Sigma-Aldrich. Ethanol and aluminium oxide (90, active neutral, powder) were purchased from Merck. Copper(I) bromide was washed with ethanol before use. Doxorubicin hydrochloride was purchased from Upjohn and used as received.

### Example 2: Quantification of Total Hydroxyl Content in Lignin Via Acetylation

The total content of hydroxyl groups including aliphatic and phenolic ones was determined. Briefly, 400 mg of dried lignin (0.08 mmol) was dissolved in 16 mL of pyridine/acetic anhydride (1:1, v/v). The reaction solution was stirred for 48 h under nitrogen at room temperature. After that, the reaction solution was added dropwise with stirring to 300 mL of ice-water. The precipitated acetylated lignin was collected by centrifugation, washed 3 times with ice-water (100 mL), freeze-dried and further dried under vacuum at 50° C. By comparing the ¹H NMR spectra of acetylated lignin with that of external reference (ethyl acetate), the content of hydroxyl groups in lignin was calculated to be 37.05 mole of —OH in one mole of lignin (aromatic —OH 40.2% and aliphatic —OH 59.8%, respectively).

### Example 3: Macro-Initiator Synthesis

Lignin-based macro-initiators are generally denoted as MI-x % (e.g. MI-5%), whereas x %, also known as degree of substitution (DS), represents the percentage of lignin hydroxyl groups that was modified into macro-initiation sites (—Br).

Typically, lignin (5.0 g, 1.0 mmol, containing 37.05 mmol of hydroxyl groups) was dried in a Schlenk flask at 90° C. under vacuum overnight. The dried lignin was cooled down to room temperature under nitrogen atmosphere. Subsequently, anhydrous DMA (50 mL) was injected into the flask to dissolve the lignin under rapid stirring. To the solution was added dropwise a corresponding amount of 2-bromoisobutyryl bromide (BiBB) dissolved in 15 mL of DMA over a period of 2 hours. The mixture was allowed to react at room temperature under nitrogen atmosphere for 48 hours. The reaction mixture was precipitated from diethyl ether, and the precipitate was dried and re-dissolved in DMF. The product was subsequently precipitated again from deionized water for removal of unreacted 2-bromoisobutyryl bromide. The product was freeze-dried for 48 hours to give the final macro-initiators.

### Example 4: Copolymerization

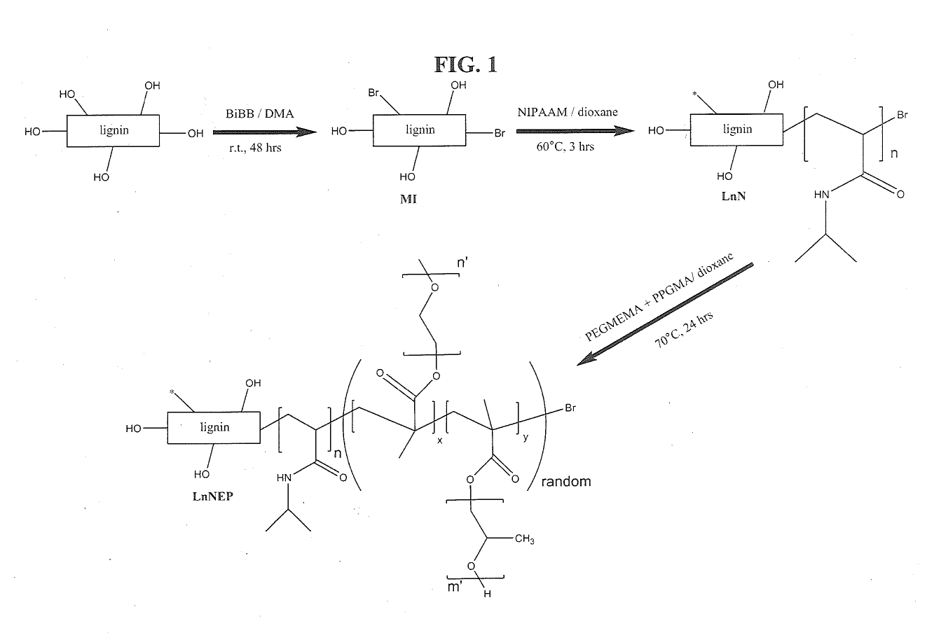

The copolymerization was a two-step ATRP reaction as shown in FIG. 1.

For the first step, lignin macro-initiator MI-40% (16.0 mg, 2.2×10⁻³ mmol) and NIPAAM (565 mg, 5 mmol) were added into 3 mL of 1,4-dioxane in a dry Schlenk flask. The flask was then sealed, repeatedly degassed and purged with nitrogen. The degassed macro-initiator/NIPAAM mixture was stirred at room temperature for 30 min. Cu(I)Br (7.2 mg, 0.05 mmol) and 1,1,4,7,10,10-hexamethyltriethylenetetramine (HMTETA) (9.2 mg, 0.04 mmol) were mixed to form Cu¹-HMTETA complex which was added into the macro-initiator/NIPAAM mixture. The flask was then degassed and stirred under nitrogen further for 30 min before being heated at 60° C. in an oil bath. The reaction was allowed to take place for 3 hours. The reaction mixture was cooled down and then passed through a neutral alumina column with 40 mL of THF as eluent. After being concentrated using a rotary evaporator, the solution was poured into excess amounts of diethyl ether to precipitate the polymer (LnN-40%).

For the second step, ATRP was initiated by the product of the first step (LnN-40%). LnN-40% (obtained from the first step ATRP), PEGMEMA (Mₙ=475, 380 mg, 0.8 mmol), and PPGMA (Mₙ=375, 188 mg, 0.5 mmol) were added into 4 mL of 1,4-dioxane in a dry 100-mL Schlenk flash. The flask was then sealed, repeatedly degassed and purged with nitrogen. Cu¹-HMTETA complex (identical to the amount used in the first step) was added into the stirring mixture. The flask was then degassed and stirred under nitrogen further for 30 min before being heated at 70° C. in an oil bath. The reaction was allowed to take place for 24 hours. The final polymer product (LnNEP-40%) was purified according to the same procedures used for the first step.

### Example 5: Molecular Characterization

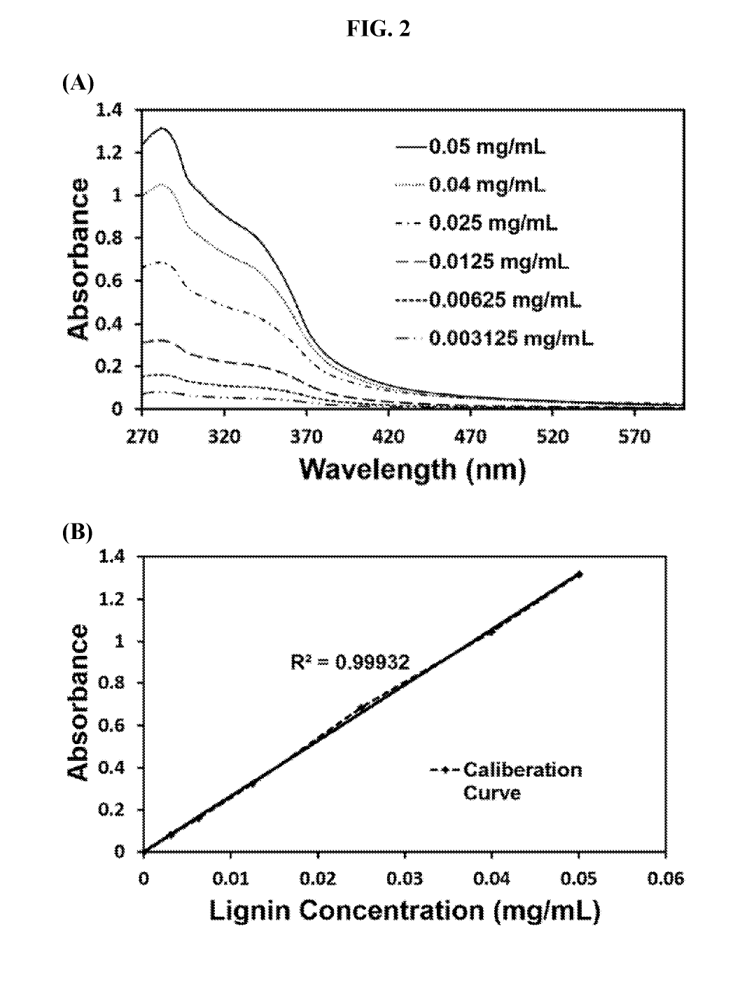

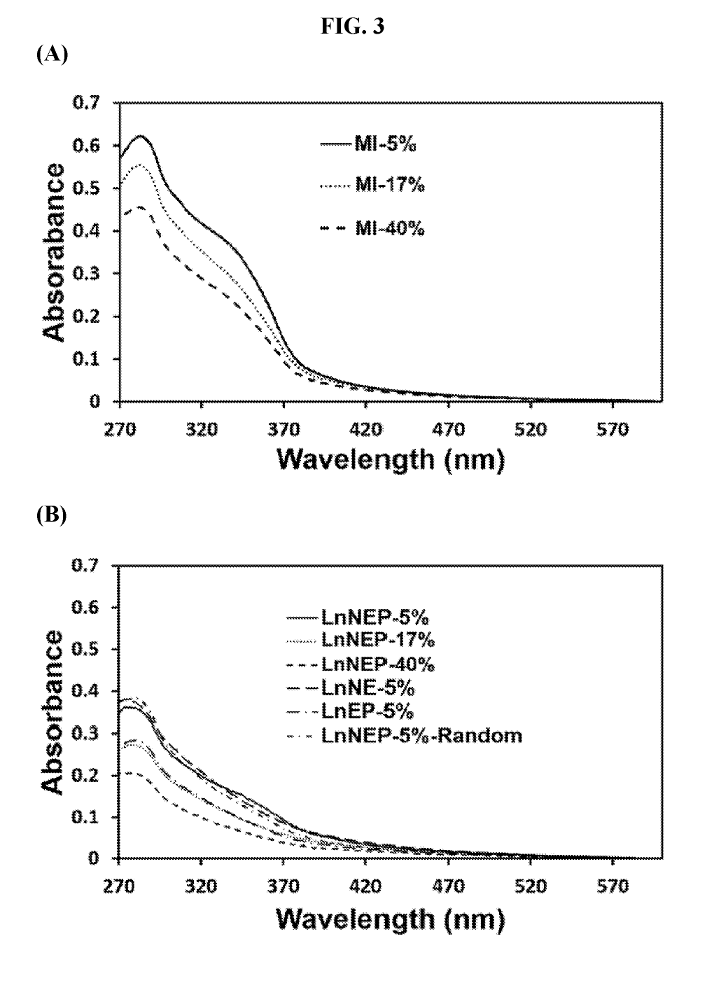

¹H NMR spectra were recorded using Bruker Avance DRX 400 NMR spectrometer. Samples (10 mg) were freeze-dried and dissolved in 0.5 mL of chloroform-d (CDCl₃). The NMR spectra were recorded at 23° C. with an acquisition time of 3.15 s. Lignin content was determined by UV spectrophotometry. Lignin was dissolved in anhydrous DMF and was subsequently diluted to various concentrations. UV-vis absorption measurements were conducted on UV-vis spectrophotometer (Shimadzu, Kyoto) in the range of 270-600 nm. A calibration curve was obtained by plotting the peak absorption values at 282 nm against known lignin concentrations (FIG. 2(b)).

### Example 6: Particle Size Measurements

Particle size and size distribution were determined by dynamic light scattering (DLS) using a Zetasizer Nano ZS (Malvern Instruments, Southborough, Mass.) with a laser light wavelength of 633 nm at 173° scattering angle. Polymer solutions (500 mg/L) were passed through a 0.45 μm pore-sized PVDF filter before measurements. Each analysis was done in duplet at pre-determined temperatures and the Z-average hydrodynamic diameters of the particles were shown. For accuracy purposes, polymer solutions were equilibrated at above-mentioned temperatures for 30 min between the measurements. Three parallel analyses were carried out and the average reading was reported.

### Example 7: Critical Aggregation Temperature (CAT)

Critical Aggregation Temperature (CAT) was determined by investigating particle size changes at different temperatures. Polymer solutions (500 mg/L) were prepared and subsequently tested using dynamic light scattering (DLS) at different temperatures ranging from 22-40° C. with 3° C. increment for each step. The CAT value was determined by the plot of the Z-average hydrodynamic diameters of the particles versus the temperatures.

### Example 8: Sol-Gel Transition

The thermo-responsive sol-gel transition of the copolymer aqueous solutions was measured by the test tube inverting method. Sample solutions at intended concentrations were prepared by dissolving corresponding amounts of polymers in distilled water in a 1.5-mL glass vial. The polymer solutions were subsequently equilibrated at 4° C. for 24 hours. The sample containing vials were immersed in a water bath at designated temperature for 15 min. The temperature was increased 0.5° C. per step. The gelation temperature was characterized by the temperature at which the solution formed a gel that remained intact when the tube was inverted by 180°. The critical gelation concentration (CGC) is defined as the minimum copolymer concentration in aqueous solution at which the gelation is observed.

### Example 9: Dynamic Rheological Studies

The sol-gel transition behavior was investigated by dynamic rheological experiments conducted on an RS600 rheometer (ThermoHaake, Waltham, Mass.). The aqueous solution of the polymer was placed between parallel plate of 35 mm in diameter and a gap of 0.5 mm. The data were collected under a controlled stress of 2.0 dyn/cm² and a frequency of 1.0 rad/s. The heating rate was 0.5° C./min. Water is added to the surrounding of the parallel plate to minimize the impact of water evaporation during data collection. Critical gelation temperature (CGT) is defined as the temperature at which G′ crosses over G″ in temperature sweep.

### Example 10: Drug Release

An aqueous solution containing 7 wt % of the copolymer was prepared and allowed to equilibrate overnight at 4° C. to form a polymer solution. Doxorubicin was mixed with the polymer solution to at the concentration of 0.1 mg/mL, followed by stirring overnight at 4° C. The resulting mixture (600 μL) was transferred into a fabric bag and immersed into a test tube containing 12 mL of phosphate buffer saline (PBS), and incubated at 37° C. At a predetermined time interval, 1.0 mL of the buffer was extracted and replaced with fresh PBS buffer. Each test was done in duplicate. The concentrations of doxorubicin in the buffer were determined by measuring the absorption at 480.5 nm.

### Example 11: Synthesis and Characterization of Copolymers

Lignin, as a natural polyhydroxyl aromatic heteropolymer, contains many hydroxyl groups which may be readily modified by 2-bromoisobutyrl bromide through esterification reaction into macro-initiation sites for ATRP reaction. In this study, the alkali kraft lignin was used, which was determined to contain 37.05 mmol of hydroxyl group (—OH) per lignin molecule. Three macro-initiators of different degree of substitution (DS) were synthesized by varying the amount of 2-bromoisobutyrl bromide (FIG. 1 and TABLE 1).

The lignin-based thermo-responsive copolymers were designed and synthesized through ATRP reaction using the lignin-based macro-initiators and three monomers NIPAAM, PEGMEMA, and PPGMA (FIG. 1).

The ATRP reaction was carried out in two steps: first grafting NIPAAm monomers onto lignin core, and then extending the grafts with PEGMEMA and PPGMA monomers. Because of the living nature of the ATRP reaction, the synthesized lignin-based copolymers, denoted as LnNEP, are composed of a lignin core and multiple arms of graft polymer chains, where each graft consists of a block of PolyNIPAAm (PNIPAAm) and a block of brush-like random copolymer of PEG/PPG. By varying the type of macro-initiators used while fixing the monomer ratio in the polymerization reactions, LnNEP copolymers with different lignin contents and numbers of PNIPAAM/PEG/PPG grafts may be obtained, but with similar graft structure and length (TABLE 2).

Control polymers LnNE-5%, LnEP-5%, and LnENP-5%-Random were also synthesized. For LnNE-5% and LnEP-5%, PEGMEMA and only one type of temperature responsive monomer (NIPAAM or PPGMA) were used. The amount of PEGMEMA and the total amount of temperature responsive monomer (NIPAAM or PPGMA) used were the same as LnNEP-5%. For example, LnNEP-5% was synthesized with 565 mg of NIPAAM, 381 mg of PEGMEMA, and 188 mg of PPGMA, while LnNE-5% was synthesized with 565+188=753 mg of NIPAAM and 381 mg of PEGMEMA. LnNEP-5%-Random was synthesized with the same types and same amounts of monomers as LnNEP-5%, but all three monomers were randomly polymerized.

TABLE 1
Synthesis of macro-initiators (MIs) with different degree of
substitution (DS) and chemical compositions

| | Feeding | | Macro-initiator | | | |
|---|---|---|---|---|---|---|
| | Lignin | | | Lignin | | |
| Macro- | in gram/ | BiBB in mL/ | | content | DS | |
| initiators^{a} | in mmol | in mmol | Yield (g) | (%)^{b} | (%)^{b} | Mₙ^{c} |
| MI-5% | 5.0/1.0 | 0.92/7.4 | 4.901 | 94.4 | 5.3 | 5300 |
| MI-17% | 5.0/1.0 | 1.83/14.8 | 4.897 | 84.1 | 17 | 5950 |
| MI-40% | 5.0/1.0 | 3.05/24.7 | 4.795 | 69.1 | 40 | 7240 |

^{a}Lignin-based macro-initiators are generally denoted as MI-x %, whereas x %, known as degree of substitution (DS), represents the percentage of lignin hydroxyl groups that was modified into macro-initiation sites.
^{b}Lignin content and actual DS were estimated from the UV absorption spectra. Therefore, the number of hydroxyl groups converted to initiation sites was 2, 6, and 15 for MI-5%, MI-17%, and MI-40%, respectively.
^{c}Estimated molecular weight of MIs was calculated from lignin content and DS.

TABLE 2
Lignin-based copolymers with different chemical compositions and
their synthesis conditions

| | MI | | | | | | | |
|---|---|---|---|---|---|---|---|---|
| | | —Br | | | | Copolymers | | |
| | MI | amount in | | | | | Lignin | |
| Lignin-based | amount | MI | Feeding of monomers (mg) | | | Yield | content | |
| copolymers^{a} | (mg) | (mmol) | NIPAAM | PEGMEMA | PPGMA | (mg) | (%)^{b} | Mₙ^{c} |
| LnNEP-5% | 53.4 | 0.020 | 565 | 381 | 188 | 564 | 10.8 | 46300 |
| LnNEP-17% | 26.7 | 0.028 | 565 | 381 | 188 | 572 | 8.2 | 61120 |
| LnNEP-40% | 16.0 | 0.033 | 565 | 381 | 188 | 574 | 6.1 | 81700 |
| LnNE-5% | 53.4 | 0.020 | 753 | 381 | 0 | 541 | 11.5 | 43400 |
| LnEP-5% | 53.4 | 0.020 | 0 | 381 | 753 | 568 | 8.6 | 58200 |
| LnNEP-5%- | 53.4 | 0.020 | 565 | 381 | 188 | 546 | 11.5 | 43400 |
| Random | | | | | | | | |

^{a}Targeted lignin-based copolymers are denoted LnNEP-x %, where Ln, N, E, and P represent lignin, NIPAAM, PEG, and PPG, respectively, and x % is the corresponding DS of the MI used. LnNE-5%, LnEP-5%, LnNEP-5%-Random were synthesized as control copolymers. All copolymers were synthesized with fixed amounts of ligand (9.2 mg) and CuBr (7.2 mg).
^{b}Lignin content was estimated from the UV absorption spectra.
^{c}Estimated molecular weight was computed from lignin content and lignin average molecular weight, which is 5000.

The lignin content in terms of mass percentage in both macro-initiators (TABLE 1) and copolymer samples (TABLE 2) was estimated from the UV absorption of lignin, as follows.

FIG. 2(a) shows the plot of UVvis spectra of lignin samples with known concentrations. A calibration curve was obtained by plotting the lignin peak absorbance at 282 nm versus the lignin concentration (FIG. 2(b)).

The calibration curve gives the linear equation as follows:

$$Lignin Concentration ( mg / mL ) = \frac{Peak Absorbance at 282 nm}{26.397}$$

The UV-vis spectra of both macro-initiators (MIs) and copolymers are shown in FIG. 3. The peak absorbance data at 282 nm are listed in TABLE 3.

TABLE 3
UV absorbance data for lignin macroinitiators (MIs), lignin-
based copolymers (LnNEPs) and control copolymers.

| | Peak Absorbance | Lignin |
|---|---|---|
| Sample Name | Value at 282 nm | Content (%) |
| MI-5% | 0.623 | 94.4 |
| MI-17% | 0.555 | 84.1 |
| MI-40% | 0.456 | 69.1 |
| LnNEP-5% | 0.355 | 10.8 |
| LnNEP-17% | 0.270 | 8.18 |
| LnNEP-40% | 0.202 | 6.12 |
| LnNE-5% | 0.380 | 11.52 |
| LnEP-5% | 0.283 | 8.58 |
| LnNEP-5%- | 0.378 | 11.51 |
| Random | | |

The lignin content was computed from the equation mentioned above, which was obtained from the calibration curve.

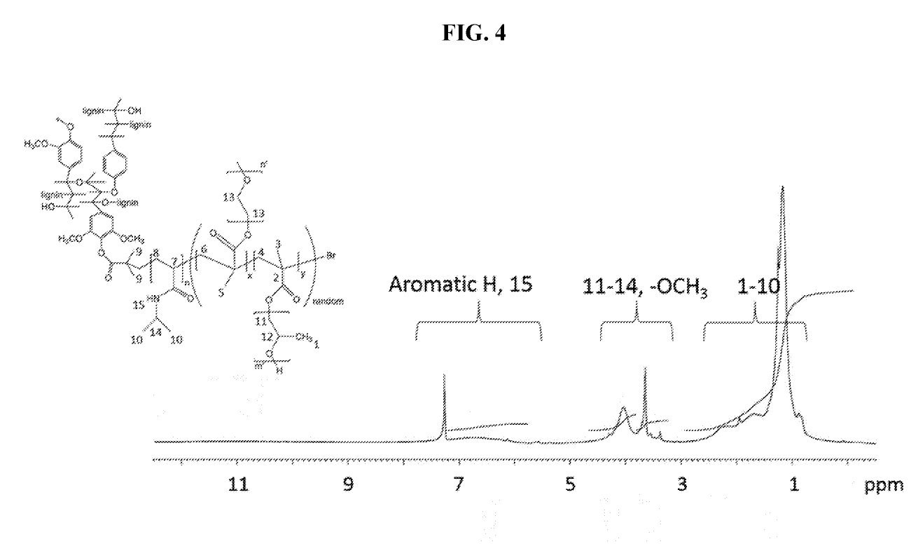

The chemical structure of LnNEP copolymer was verified by ¹H NMR spectroscopy. FIG. 4 shows the ¹H NMR spectrum of LnNEP-5% in CDCl₃, in which proton signals belonging to PNIPAAM, PEG and PPG portions are all confirmed. The sharp signals at around 1 ppm are assigned to the protons of the methyl groups in PPG and PNIPAAm. The signals at 1.5-2.5 ppm are from the protons of the graft copolymer backbones. The signals from 3-4.5 ppm are attributed mainly to protons of PEG and PPG backbones, and —CH next to the amide group, as well as —OCH₃ in lignin. The signals at 5.5-7.5 ppm correspond to protons of the aromatic rings in lignin and the protons that are attached to nitrogen atom.

### Example 12: Thermo-Responsive Behavior of Lignin-Based Copolymers

The LnNEP copolymers were water-soluble at room temperature and could form aggregations at elevated temperatures depending on the copolymer architectures and compositions. The copolymers contain the lignin core that is hydrophobic, the PEG segments that are hydrophilic, and PPG/NIPAAM segments that are temperature-responsive and can transit from hydrophilic to hydrophobic when temperature is increased.

For example, PNIPAAm has its lower critical solution temperature (LCST) at 32° C. PPG, on the other hand, exhibits a molecular weight and structure-dependent LCST in the range of 14-100° C. As the temperature of the lignin-based copolymer solutions is increased, the PPG segments behaves more favorably as hydrophobic components, while the PNIPAAm segments would experience a transition of hydrophobicity at 32° C. The coupled thermo-responsive effect of PPG and PNIPAAm together with the effect of the lignin core and PEG segments that adjust the hydrophilicity/hydrophobicity balance would impart the copolymers with interesting thermo-responsive properties.

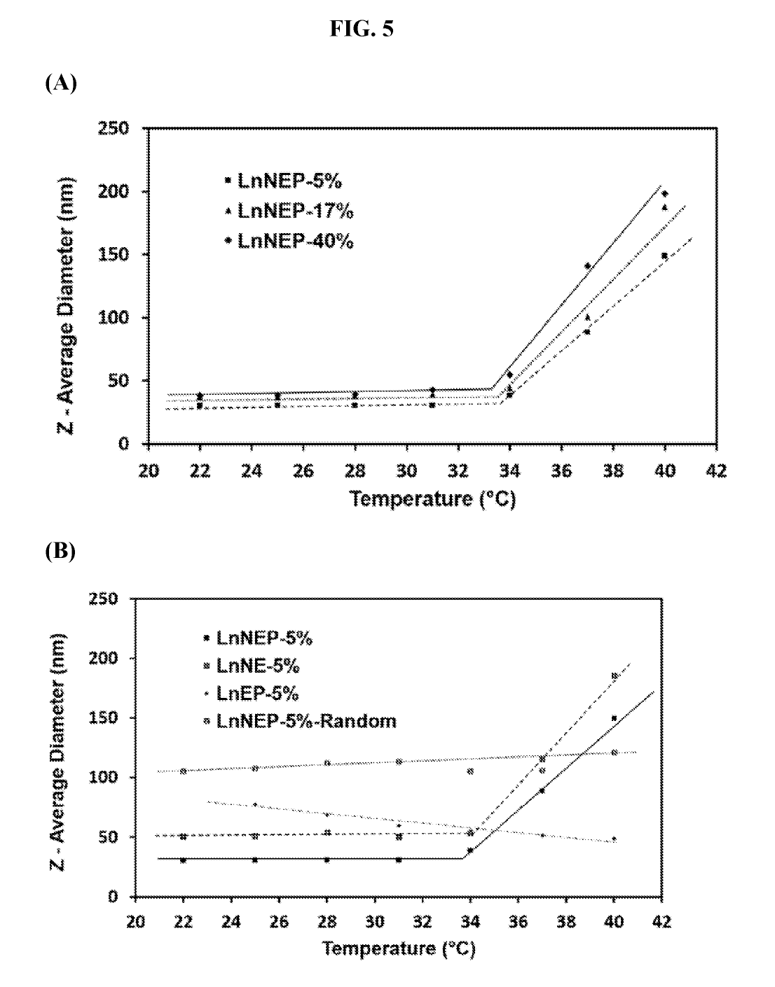

TABLE 4 shows the average diameters and polydispersity indexes of the different lignin-based copolymers dissolved in water at 25 and 37° C. Further, in FIG. 5, the average diameters of the lignin-based copolymers are plotted against different temperatures. The average diameters of LnNEP-5%, LnNEP-17%, LnNEP-40%, and LnNE-5% showed a sudden increase at around 33-34° C., indicating that these copolymers formed aggregations above a critical temperature, which is the critical aggregation temperature (CAT), as listed in TABLE 4. All these four copolymers contained the block structure of PNIPAAm. LnEP-5%, which did not contain PNIPAAm segments, did not show a sudden change in its average diameter. Although LnEP-5% contained PPG segments that are thermo-responsive, its short length only containing about 6 propylene glycol units corresponds to a very high transition temperature. Moreover, even though LnNEP-5%-Random contained PNIPAAm segments randomly dispersed along the polymer chain, it did not display any thermo-responsive aggregation, due to the lack of PNIPAAm block structure.

TABLE 4
Particle size and PDI data for lignin-based copolymers in water.

| | Z-average | | Polydispersity | | |
|---|---|---|---|---|---|
| Copolymer | diameter (nm) | | index (PDI) | | CAT |
| sample^{a} | 25° C. | 37° C. | 25° C. | 37° C. | (° C.)^{b} |
| LnNEP-5% | 31 | 84 | 0.200 | 0.038 | 33.2 |
| LnNEP-17% | 37 | 101 | 0.242 | 0.032 | 33.6 |
| LnNEP-40% | 40 | 198 | 0.238 | 0.008 | 33.8 |
| LnNE-5% | 51 | 185 | 0.233 | 0.082 | 34.0 |
| LnEP-5% | 77 | 51 | 0.335 | 0.234 | — |
| LnNEP-5%- | 107 | 121 | 0.445 | 0.347 | — |
| Random | | | | | |

^{a}Concentration at 0.5 mg/mL.
^{b}Obtained from FIG. 5.

### Example 13: Thermogelling Property of Lignin-Based Copolymers

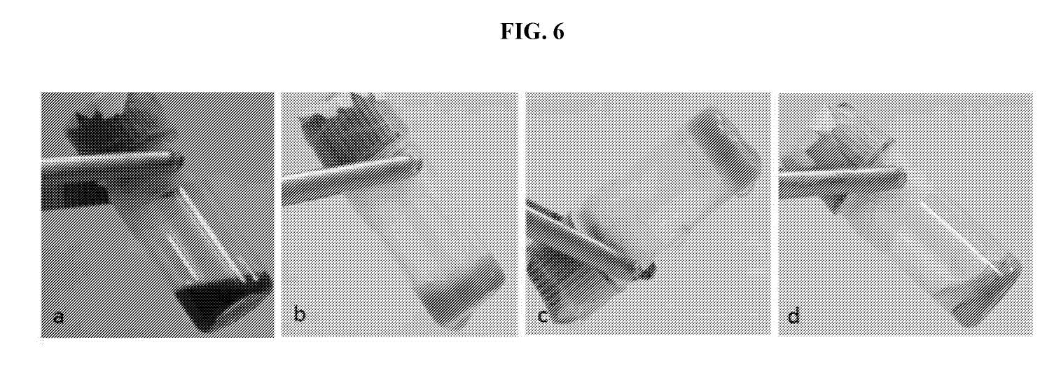

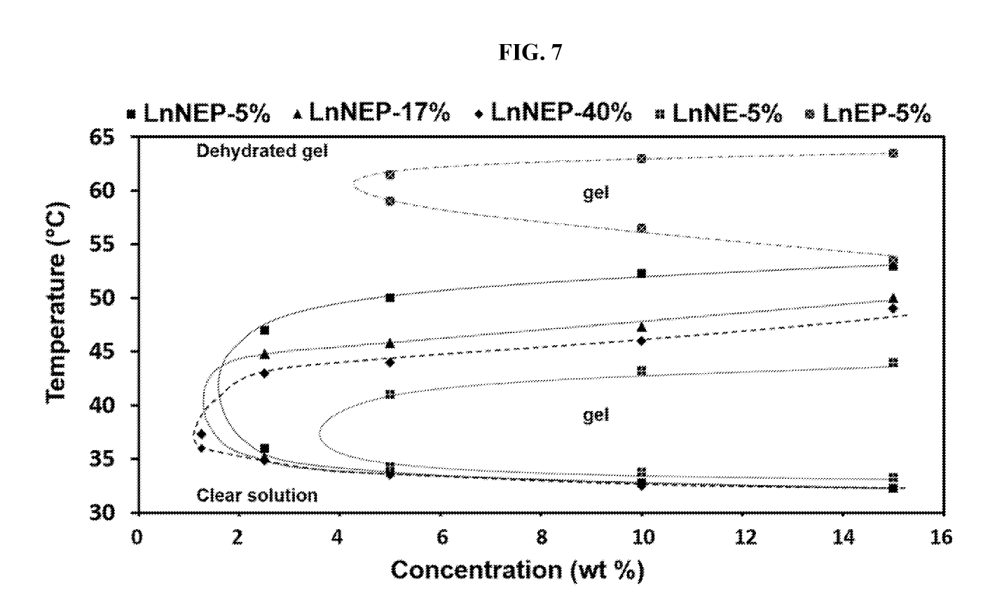

The solutions of all three LnNEP copolymers underwent a sol-gel-dehydrated gel transition upon increasing temperature. Taking LnNEP-5% (7.0 wt %) as example, as shown in FIG. 6, the polymer solution behaved as a liquid at room temperature. Upon reaching 33° C., the polymer solution changed to semi-solid state, and turned from transparent to turbid. By raising the temperature further to 35° C., the polymer solution formed a firm hydrogel, and stayed intact upon vial inversion. At 50° C., the gel deswelled, expelling its water content, and reached a dehydrated state. It is thought that at 50° C., the hydrophobic interactions between PNIPAAM blocks and/or PPG segments were so strong that the LnNEP-5% copolymer hydrogels shrank in size, and expelled the water molecules from the hydrogel structure. The phase diagrams of the aqueous polymer solutions were determined by the test tube inverting method that we reported earlier, and the results are illustrated in FIG. 7. The critical gelation concentration (CGC) for LnNEP copolymers was between 1.3 (for LnNEP-40%) to 2.5 wt % (for LnNEP-5%). These values are much lower than that of the commercially available Pluronic F127 copolymer, which is about 17 wt %.

When comparing LnNEP-5% and LnNE-5%, although they both formed hydrogels at a similar temperature (about 33° C.), the former had a lower CGC and could remain to be hydrogel at much higher temperature up to about 50° C. Both copolymers were synthesized with MI-5% macro-initiator and they had similar molecular weight. However, LnNEP-5% contained both PNIPAAm and PPG as its thermo-responsive segments, while LnNE-5% had only PNIPAAm but zero PPG (here the mass of PNIPAAm in LnNE-5% is equivalent to that of PNIPAAm plus PPG in LnNEP-5%). It is believed that the PNIPAAm block is essential for the copolymers to demonstrate a lower gelation temperature at about 33° C., which is around the LCST of PNIPAAm homopolymer. For the case of LnNEP-5%, the PPG segments would assist in water retention by balancing the hydrophilicity/hydrophobicity of the hydrogel network at the temperature range up to about 50° C. It was reported that PPG (425 Da) showed LCST at 52° C. In our lignin-based copolymers the PPG chain length is about 300 Da, so its LCST should be higher than 52° C., implicating that the PPG segments in LnNEP-5% are hydrophilic below its LCST, which made the dehydration temperatures of LnNEP-5% higher than 50° C. In the meantime, for LnNE-5% where the PPG are replaced by PNIPAAm, the hydrogels dehydrated at lower temperatures which are around 43° C. This is consistent with that chemically crosslinked PNIPAAm hydrogels dehydrated and lost about 90% of the water in the hydrogels at 40° C. and above.

For LnEP-5%, it required a very high temperature up to 55° C. or above for the hydrogel formation due to the lack of PNIPAAm block. It can be understood because the LCST of the PPG segments in LnEP-5% is at least as high as 52° C. Finally, LnNEP-5%-Random, where the NIPAAm units are randomly dispersed with PEG/PPG segments, was not able to form hydrogels at the temperature ranges tested, although it had the same chemical composition as LnNEP-5%. The solution of LnNEP-5%-Random only turned from transparent to turbid when the temperature was increased. The results further confirmed that the PNIPAAm block is essential for the copolymers to form hydrogels at a temperature around the LCST of PNIPAAm polymer.

### Example 14: Thermo-Responsive Rheological Properties

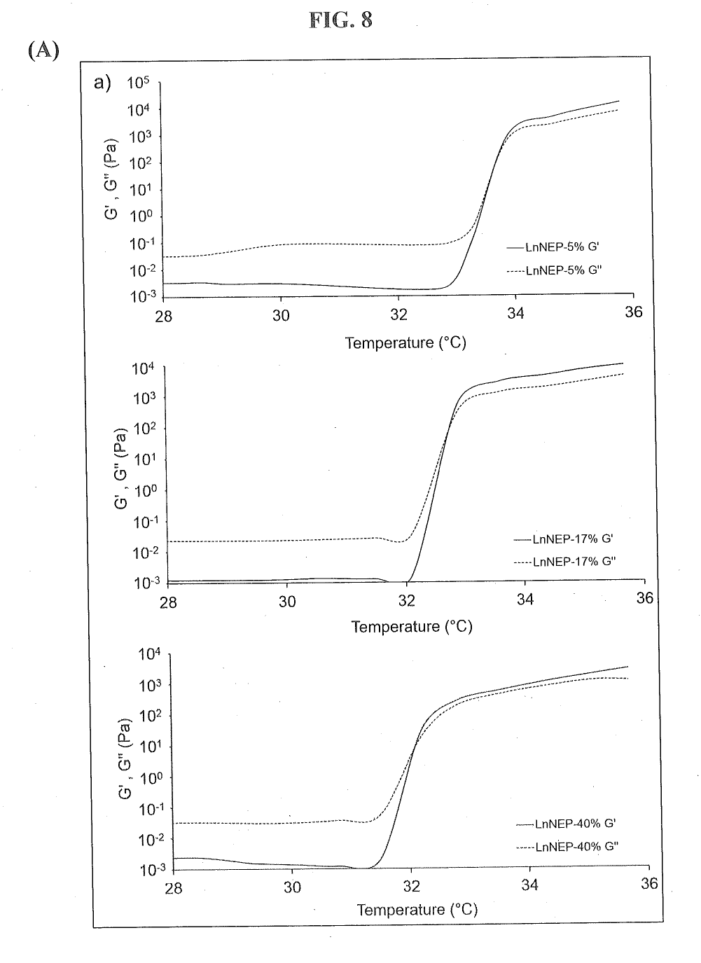

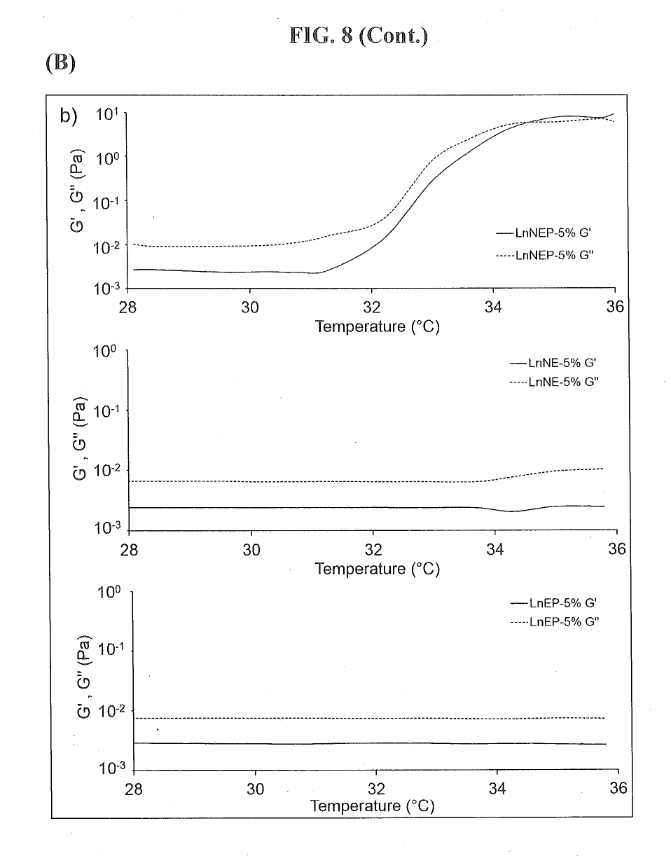

The rheological properties of the thermo-responsive copolymers and their hydrogels were studied. FIG. 8(A) shows how the storage modulus G′ and the loss modulus G″ of the LnNEP copolymers at 7.0 wt % were influenced by temperature.

At low temperatures, both G′ and G″ of these copolymer solutions were very low, with G′ being much lower than G″, indicating that all the copolymer solutions existed as liquids. As temperature increased to the range of about 31.5-33° C., their G′ and G″ both began to increase dramatically, with G′ increasing much faster than G″, giving a crossover point, beyond which the G′ was higher than G″, indicating that the copolymer solutions formed hydrogels at the temperature of the crossover point. This onset temperature is also known as the critical gelation temperature (CGT). The CGT values obtained from FIG. 8(A) are 33.7, 32.8, and 32.1° C. for LnNEP-5%, LnNEP-17%, and LnNEP-40%, respectively. It is clear that all LnNEP copolymer solutions showed thermogelling transition at a temperature above room temperature but below human body temperature, suggesting that the hydrogels are potentially useful in biomedical applications such as for injectable controlled drug release, or stem cells culture and differentiation. Furthermore, the G′ of the hydrogels could be in the range of 2700-13900 Pa, by simply changing the lignin-based macro-initiators used.

FIG. 8(B) shows a comparison of LnNEP-5% with LnNE-5% and LnEP-5% at 2.5 wt % in terms of their rheological properties at different temperatures. Both G′ and G″ of LnNEP-5% showed remarkable increase after the temperature reached 31° C., and then the G′ and G″ reached the crossover point at the CGT (34.6° C.), forming a hydrogel. However, the G′ and G″ of LnNE-5% and LnEP-5% remained unchanged at very low levels throughout the temperature range tested. LnNE-5% and LnEP-5% eventually could not form hydrogels at the concentration of 2.5 wt %, as shown in the phase diagram (FIG. 7).

### Example 15: Lignin-Based Copolymer Hydrogels for Drug Release

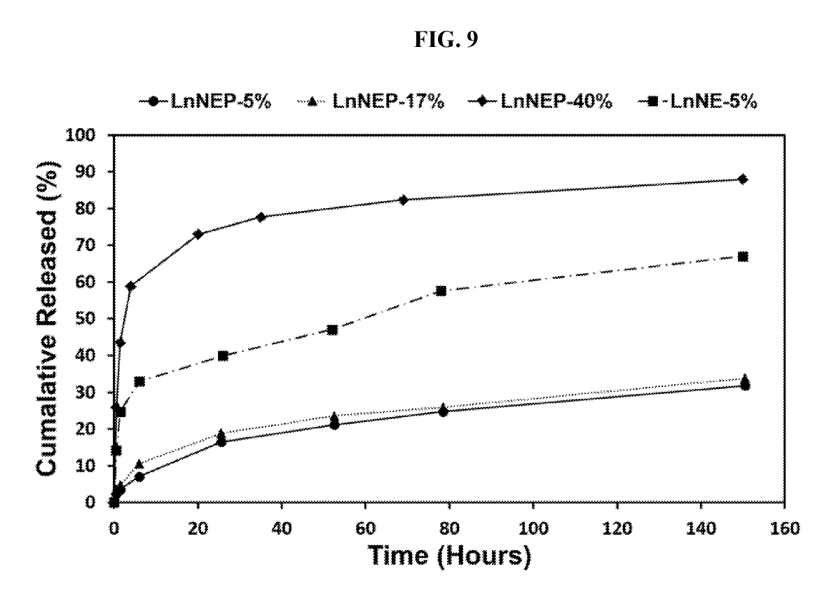

Doxorubicin (DOX) release profiles from the lignin-based copolymer hydrogels in phosphate buffer saline (PBS) are illustrated in FIG. 9. The lignin-based copolymers synthesized from different macro-initiators demonstrated very different release profiles. The hydrogels of LnNEP-5% and LnNEP-17% showed more sustained release than those of LnNEP-40%. LnNE-5% gave release kinetics in between of those hydrogels, although LnNE-5% had higher CGC and smaller gelation window in the phase diagram than others. In the rheological property studies (FIG. 8(A)), LnNEP-5% and LnNEP-17% showed much higher G′ than that of LnNEP-40%. The much stronger hydrogels of LnNEP-5% and LnNEP-17% could result in much slower release of the drug.

### Example 16: Further Experimental Results

TABLE 5 demonstrates thermo-responsive rheology data. The data were collected under a controlled stress of 2.0 dyn/cm² and a frequency of 1.0 rad/s. The heating rate was 0.5° C./min.

TABLE 5
Rheological Characteristics of Hydrogels of
LnNEP Copolymers at 7 wt % Concentration

| Sample | Concentration | | | |
|---|---|---|---|---|
| Name | (wt %) | G′ (Pa)^{a} | G″ (Pa)^{a} | CGT (° C.)^{b} |
| LnNEP-5% | 7.0 | 13870 | 6597 | 33.7 |
| LnNEP-17% | 7.0 | 9112 | 4234 | 32.8 |
| LnNEP-40% | 7.0 | 2722 | 1159 | 32.1 |

^{a}G′, G″ values recorded at 35.7° C.
^{b}Critical gelation temperature (CGT) determined at the crossover point of G′ and G″ in temperature sweep.

### Example 17: Summary

A series of thermogelling copolymers, LnNEP-5%, LnNEP-17%, and LnNEP-40%, were synthesized from lignin-based macro-initiators, NIPAAm, PEGMEMA, and PPGMA through a two-step ATRP living polymerization. A few control copolymers, LnNE-5%, LnEP-5%, and LnNEP-5%-Random, were also synthesized for comparison purpose. It was found that all LnNEP copolymers were thermo-responsive in aqueous medium. The aqueous solutions of the copolymers showed thermo-responsive aggregation at a very low concentration (0.50 mg/mL) at their CATs of around 33-34° C., which were very close to the LCST of PNIPAAm (32° C.). The aqueous solutions of LnNEP copolymers displayed thermogelling behaviors, turning from sol at low temperatures to hydrogel at CGT around 32-34° C., and further to dehydrated gel at higher temperatures. The thermogelling LnNEP copolymers had very low CGCs ranging from 1.3 to 2.5 wt %. It is believed that in the LnNEP copolymers, the PNIPAAm block is essential to the gelation at the CGTs, while the PPG segments would assist in water retention by balancing the hydrophilicity/hydrophobicity of the hydrogel network at the temperature range up to about 50° C. The LnNEP copolymers showed thermoresponsive rheological properties. The G′ and G″ of the copolymer solutions were very low at low temperatures, and increased at higher temperatures, and then crossed over at their gelation temperatures, with G′ dominating G″ after the gel formation. The G′ of the hydrogels could be tuned in the range of 2700-13900 Pa by changing the types of lignin-based macro-initiators.

The LnNEP copolymer hydrogels were tested for doxorubicin release in PBS. The hydrogels of LnNEP-5% and LnNEP-17%, which showed higher G′ than LnNEP-40%, released the drug at much slower rate. The LnNEP copolymers, showing thermogelling transition at a temperature above room temperature but below human body temperature, could be potentially useful in biomedical applications such as for injectable controlled drug release.

While the present invention has been particularly shown and described with reference to exemplary embodiments thereof, it will be understood by those of ordinary skill in the art that various changes in form and details may be made therein without departing from the spirit and scope of the present invention as defined by the following claims.

1. A thermogelling graft copolymer, wherein the copolymer is obtainable by
(a) polymerizing, in a first step, an activated lignin-based polymer with N-isopropylacrylamide (NIPAAm) to form a lignin/poly(N-isopropylacrylamide) (PNIPAAm) graft copolymer, and
(b) polymerizing, in a second step, the lignin/poly(N-isopropylacrylamide) (PNIPAAm) graft copolymer with a monomer mixture comprising (i) poly(ethylene glycol) (meth)acrylate and/or a C₁₋₁₀ alkyl ether thereof, and (ii) poly(propylene glycol) (meth)acrylate and/or a C₁₋₁₀ alkyl ether thereof, to obtain a lignin-poly(N-isopropylacrylamide)-block-poly(ethylene glycol)/poly(propylene glycol) graft copolymer.

2. The thermogelling graft copolymer according to claim 1, wherein the activated lignin-based polymer is obtainable by reacting a lignin-based polymer with an atom transfer radical polymerization (ATRP) initiator.

3. The thermogelling graft copolymer according to claim 2, wherein the lignin-based polymer has a number average molecular weight in the range of about 3000 to about 7000, preferably about 5000.

4. The thermogelling graft copolymer according to claim 2, wherein the atom transfer radical polymerization (ATRP) initiator comprises or consists of 2-bromoisobutyryl bromide.

5. The thermogelling graft copolymer according to claim 1, wherein lignin is present in the lignin-poly(N-isopropylacrylamide)-block-poly(ethylene glycol)/poly(propylene glycol) graft copolymer in an amount in the range of about 5 wt % to about 15 wt %.

6. The thermogelling graft copolymer according to claim 1, wherein the thermogelling graft copolymer is represented by formula (I) 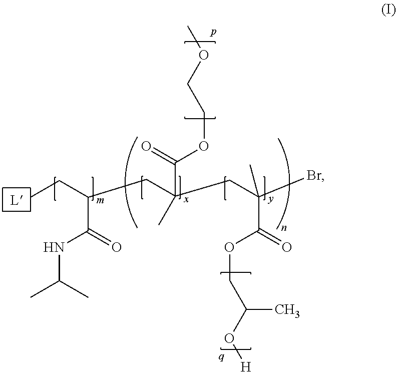
wherein L′ is the lignin-based polymer, and m, n, x, y, p and q are independently integers in the range of about 0 to 10,000.

7. The thermogelling graft copolymer according to claim 6, wherein m, n, x, y, p and q are independently integers in the range of about 5 to about 500.

8. The thermogelling graft copolymer according to claim 1, wherein the lignin-poly(N-isopropylacrylamide)-block-poly(ethylene glycol)/poly(propylene glycol) graft copolymer forms a hydrogel at a temperature in the range of about 32° C. to about 50° C.

9. The thermogelling graft copolymer according to claim 1 having a critical gelation concentration in the range of about 1 wt % to about 5 wt % in solution.

10. The thermogelling graft copolymer according to claim 1 having a critical gelation concentration in the range of about 1 wt % to about 3 wt % in solution.

11. A method of preparing a thermogelling graft copolymer according to claim 1, comprising
a) reacting a lignin-based polymer with an atom transfer radical polymerization (ATRP) initiator to form an activated lignin-based polymer;
b) polymerizing N-isopropylacrylamide (NIPAAm) with the activated lignin-based polymer by ATRP to form a lignin/poly(N-isopropylacrylamide) (PNIPAAm) graft copolymer, and
c) polymerizing the lignin/poly(N-isopropylacrylamide) (PNIPAAm) graft copolymer with a monomer mixture comprising (i) poly(ethylene glycol) (meth)acrylate and/or a C₁₋₁₀ alkyl ether thereof, and (ii) poly(propylene glycol) (meth)acrylate and/or a C₁₋₁₀ alkyl ether thereof, to obtain the lignin-poly(N-isopropylacrylamide)-block-poly(ethylene glycol)/poly(propylene glycol) graft copolymer.

12. The method according to claim 11, wherein the lignin-based polymer is dissolved in a solvent, preferably N,N-dimethylacetamide.

13. The method according to claim 11, wherein the atom transfer radical polymerization (ATRP) initiator comprises or consists of 2-bromoisobutyryl bromide.

14. The method according to claim 11, wherein between about 5% to about 40% of hydroxyl groups in the lignin-based polymer is modified into macro-initiation sites in the activated lignin-based polymer.

15. The method according to claim 11, wherein the polymerization is carried out in the presence of a catalyst comprising a complex formed from Cu(I)Br and 1,1,4,7,10,10-hexamethyltriethylenetetramine.

16. The method according to claim 11, wherein lignin content in the activated lignin-based polymer is in the range of about 65 wt % to about 95 wt %.

17. The method according to claim 11, wherein weight ratio of N-isopropylacrylamide to the monomer mixture comprising (i) poly(ethylene glycol) (meth)acrylate and/or a C₁₋₁₀ alkyl ether thereof, and (ii) poly(propylene glycol) (meth)acrylate and/or a C₁₋₁₀ alkyl ether thereof, is in the range of about 1:1 to about 2:1.

18. A composition comprising a thermogelling graft copolymer according to claim 1 and a therapeutic agent.

19. The composition according to claim 18, wherein the therapeutic agent is a nucleic acid, including DNA, a peptide, a protein, a small molecule, a cell, an antibody, an antigen, a ligand, a hormone, a growth factor, a cell signalling molecule, a cytokine, an enzyme inhibitor, an antibiotic, a chemotherapeutic agent, an anti-inflammatory agent, or an analgesic.

20. The composition according to claim 18, having a critical gelation temperature below 37° C.
